(19) United States
(12) Patent Application Publication
(10) Pub. No.: US 2018/0264280 A1
(43) Pub. Date: Sep. 20, 2018

(54) ORDERING CONDITIONER

(71) Applicant: Chih-Chung Yeh, New Taipei City (TW)

(72) Inventor: Chih-Chung Yeh, New Taipei City (TW)

(21) Appl. No.: 15/458,030

(22) Filed: Mar. 14, 2017

(51) Int. Cl. A61N 2/06

(52) U.S. Cl. CPC A61N 2/06

(57) ABSTRACT

An ordering conditioner, including: a seat body, having a connection portion, one end of the connection portion in connection with a front end of the seat body, another end of the connection portion a rear end thereof; a first bowl, configured on the front end of the seat body, a first magnetic element configured on an inner bottom of the first bowl, the first magnetic element having an N pole and S pole, the S pole of the first magnetic element in connection with the inner bottom of the first bowl; and a second bowl, configured on the rear end of the seat body, a second magnetic element configured on an inner bottom of the second bowl, the second magnetic element having an N pole and S pole, the N pole of the second magnetic element in connection with the inner bottom of the second bowl.

### (a) TECHNICAL FIELD OF THE INVENTION

The present invention relates to an ordering conditioner, and more particularly to an ordering conditioner, utilizing magnetic energy generated from first and second magnetic elements to allow medical personnel to condition patients' bodies so as to reinforce or reduce orderly energy, thereby achieving auxiliary medical conditioning.

### (b) DESCRIPTION OF THE PRIOR ART

The survival of mankind and the growth of all creatures are dependent on the continuous development and balance of ordered energy such that continuous reproduction can then breed in an endless succession. The so-called “balance” is that energy is in a satisfaction state, namely, a normal state, and if energy is insufficient, overlarge or out of order, energy imbalance, i.e. sickness, might be caused. This system of diagnosis, treatment, medical research, development and application is called ordering energy medicine; the energy medicine is first to find the cause, and then carry out the fundamental treatment, thereby avoiding long-term medication instability and the pain due to the endurance of its sequelae, complications.

Furthermore, the human body is the epitome of the whole universe such that the human body is taken as a small universe, but the universe is unlimited and the human body is limited, they all being orderly resonance systems. Therefore, there is the human body, there generates the problems of quantity and order. If humans want to live longer, their bodies must have orderly symbiosis and resonance with the universal energy.

Since the human body has limited energy, it is easier to set the quantity and order thereof; ordered energy value can be taken as a standard for the human body after we set the energy, this standard being the so-called “negative entropy”. The energy in combination with the quantity and order is orderly energy; the human body has a certain orderly energy. If the orderly energy is more than the negative entropy, that is sick, and if the energy is not enough, that is also sick. Taking magnetic energy as an example, the magnetic energy is magnetic field; the whole globe has order magnetic fields, and humans also have magnetic fields such that there will be static electricity generated if two hands are rubbed. The use of ordered magnetic use has reached the clinical application of medical disease prevention.

From a medical point of view, supplementing or extracting to adjust energy to achieve the purpose of medical care according to the amount and orderly state thereof is the so-called “ordering energy medicine”. When the body energy is out of order, this energy will be drained to keep the body energy in an orderly balance, and if the body energy is not enough, orderly energy will be made up to recover the body energy back in its original orderly balance; the energy of a certain part of the body is drained, and another part thereof will be supplemented enough so that endocrine function is normal, acid-base is balanced, positive and negative potential is balanced, and therefore, the body is healthy naturally.

### SUMMARY OF THE INVENTION

The main object of the present invention is to provide an ordering conditioner, utilizing orderly magnetic energy to provide medical personnel for conditioning patients' bodies so as to implement or extract orderly energy, thereby achieving auxiliary medical conditioning.

To achieve the object mentioned above, the present invention proposes an ordering conditioner, including: a seat body, having a connection portion, one end of the connection portion in connection with a front end of the seat body, another end of the connection portion a rear end thereof, and the connection portion being slightly narrower than the front and rear ends of the seat body; a first bowl, configured on the front end of the seat body, a first magnetic element configured on an inner bottom of the first bowl, a first filler configured between an inner side wall of the first bowl and a side of the first magnetic element, the first magnetic element having an N pole and S pole, the S pole of the first magnetic element in connection with the inner bottom of the first bowl; and a second bowl, configured on the rear end of the seat body, a second magnetic element configured on an inner bottom of the second bowl, a second filler configured between an inner side wall of the second bowl and a side of the second magnetic element, the second magnetic element having an N pole and S pole, the N pole of the second magnetic element in connection with the inner bottom of the second bowl.

According to the ordering conditioner, an outer side of the first bowl is further covered with an isolation layer.

According to the ordering conditioner, an outer side of the second bowl is further covered with an isolation layer.

According to the ordering conditioner, the first filler is made of far infrared material.

According to the ordering conditioner, the second filler is made of far infrared material.

According to the ordering conditioner, the connection portion is made of non-magnetic material.

According to the ordering conditioner, the first bowl is made of magnetic material.

According to the ordering conditioner, the second bowl is made of magnetic material.

### BRIEF DESCRIPTION OF THE DRAWINGS

FIG. 1 is a perspective view of the present invention;

FIG. 2 is a cross-sectional view of a preferred embodiment according to the present invention; and

FIG. 3 is a cross-sectional view of another preferred embodiment according to the present invention.

### DETAILED DESCRIPTION OF THE PREFERRED EMBODIMENTS

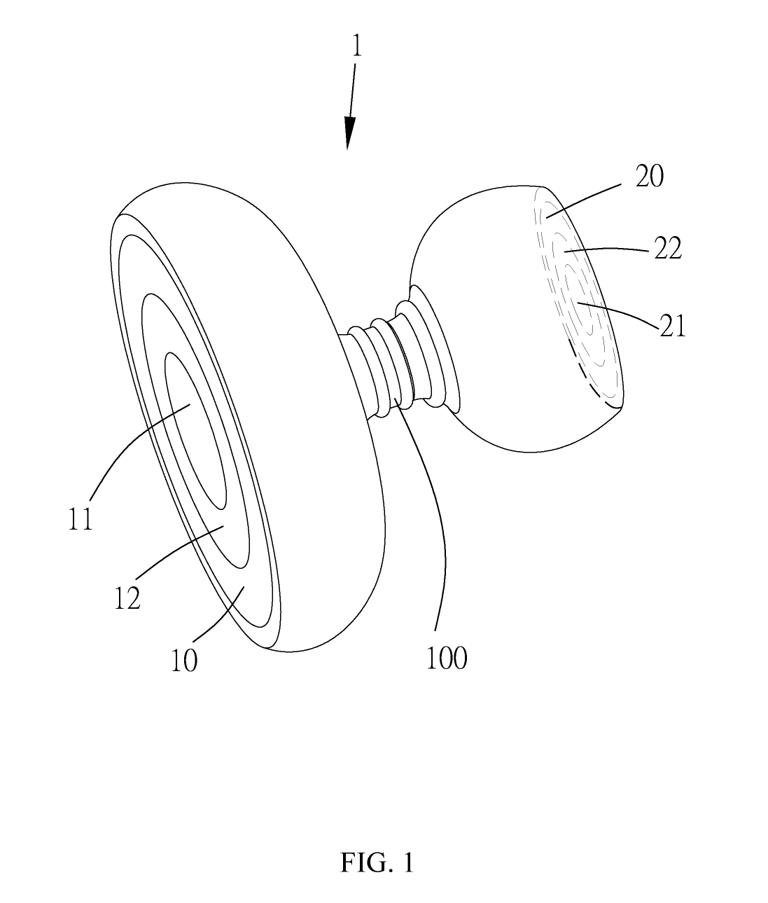

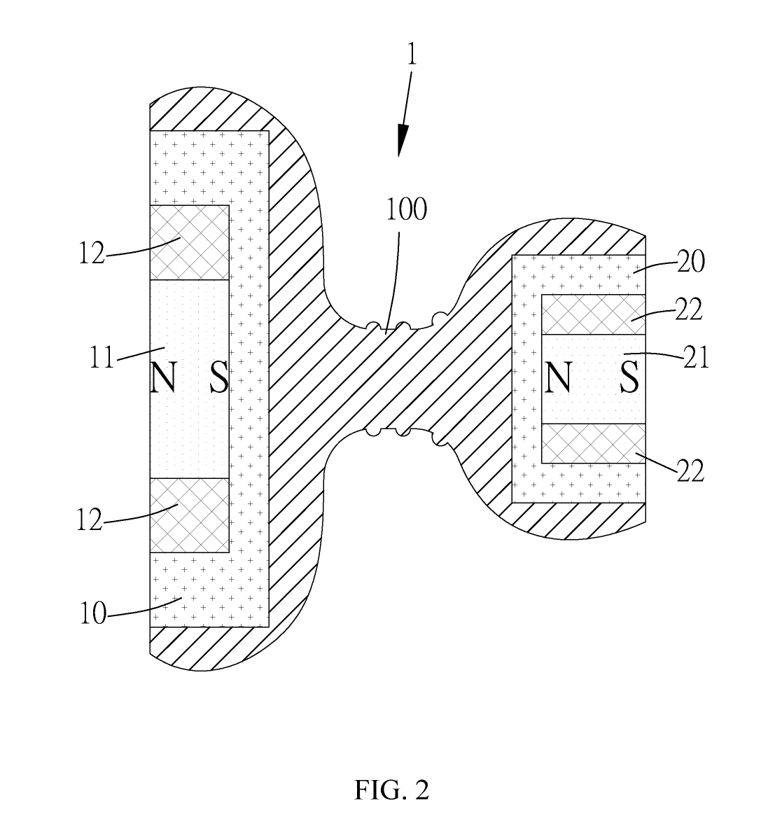

Referring to FIGS. 1 and 2, in a preferred embodiment, an ordering conditioner of the present invention includes: a seat body 1 having a connection portion 100, one end of which is in connection with the front end of the seat body 1, and another end of which the rear end thereof, with the connection portion 100 being slightly smaller than the front and rear ends of the seat body 1 in width; a first bowl 10, configured on the front end of the seat body 1, a first magnetic element 11 being configured on the inner bottom of the first bowl 10, a first filler 12 being configured between the inner side wall of the first bowl 10 and the side of the first magnetic element, and the first magnetic element 11 having an N pole and S pole, with the S pole there of being in connection with the inner bottom of the first bowl 10; and a second bowl 20, configured on the rear end of the seat body 1, configured on the rear end of the seat body 1, a second magnetic element 21 being configured on the inner bottom of the second bowl 20, a second filler 22 being configured between the inner side wall of the second bowl 20 and the side of the second magnetic element 21, and the second magnetic element 21 having an N pole and S pole, with the N pole of the second magnetic element 21 being in connection with the inner bottom of the second bowl 20, where the connection portion 100 is made of non-magnetic material, the first bowl 10 magnetic material, and second bowl 20 magnetic material, the first filler 12 and second filler 22 being respectively made of far infrared material.

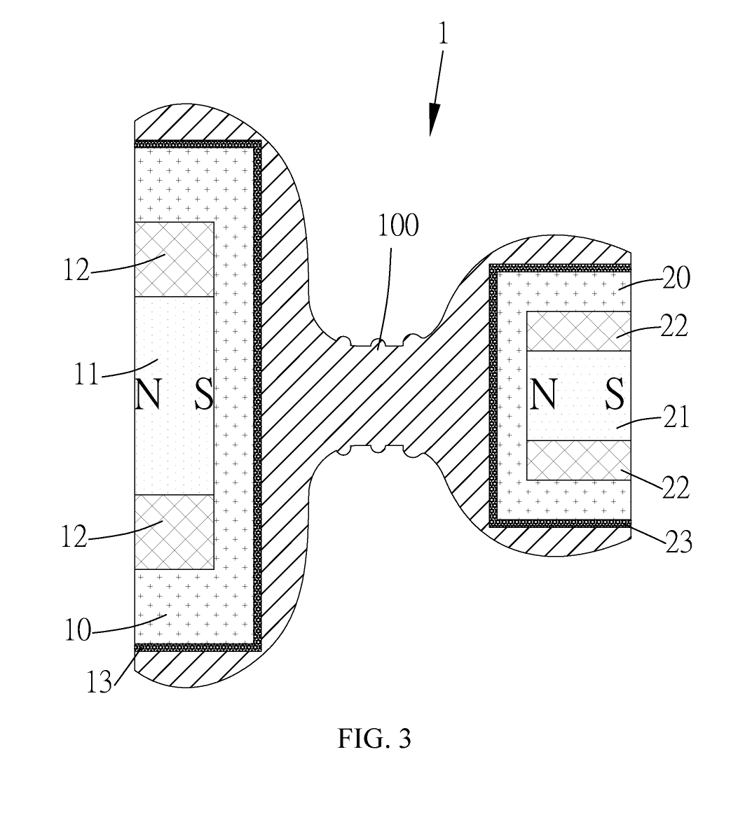

Furthermore, referring to FIG. 3, in another preferred embodiment, an ordering conditioner of the present invention includes: a seat body 1 having a connection portion 100, one end of which is in connection with the front end of the seat body 1, and another end of which the rear end thereof, with the connection portion 100 being slightly smaller than the front and rear ends of the seat body 1 in width; a first bowl 10, configured on the front end of the seat body 1, a first magnetic element 11 being configured on the inner bottom of the first bowl 10, a first filler 12 being configured between the inner side wall of the first bowl 10 and the side of the first magnetic element, the first magnetic element 11 having an N pole and S pole, with the S pole there of being in connection with the inner bottom of the first bowl 10, and the outer side of the first bowl 10 being further covered with an isolation layer 13; and a second bowl 20, configured on the rear end of the seat body 1, configured on the rear end of the seat body 1, a second magnetic element 21 being configured on the inner bottom of the second bowl 20, a second filler 22 being configured between the inner side wall of the second bowl 20 and the side of the second magnetic element 21, the second magnetic element 21 having an N pole and S pole, with the N pole of the second magnetic element 21 being in connection with the inner bottom of the second bowl 20, and the outer side of the second bowl 20 being further covered with an isolation layer 23, where the connection portion 100 is made of non-magnetic material, the first and second bowls 10, 20 respectively are made of magnetic material, and the first filler 12, 22 respectively far infrared material. Whereby, the ordering conditioner of the present invention can use orderly magnetic energy to provide medical personnel for conditioning patients' bodies so as to supplement or extract orderly energy to achieve the effects of auxiliary medical conditioning.

I claim:

1. An ordering conditioner, comprising:
a seat body, having a connection portion, one end of said connection portion in connection with a front end of said seat body, another end of said connection portion a rear end thereof, and said connection portion being slightly narrower than said front and rear ends of said seat body;
a first bowl, configured on said front end of said seat body, a first magnetic element configured on an inner bottom of said first bowl, a first filler configured between an inner side wall of said first bowl and a side of said first magnetic element, said first magnetic element having an N pole and S pole, said S pole of said first magnetic element in connection with said inner bottom of said first bowl; and
a second bowl, configured on said rear end of said seat body, a second magnetic element configured on an inner bottom of said second bowl, a second filler configured between an inner side wall of said second bowl and a side of said second magnetic element, said second magnetic element having an N pole and S pole, said N pole of said second magnetic element in connection with said inner bottom of said second bowl.

2. The ordering conditioner according to claim 1, wherein an outer side of said first bowl is covered with an isolation layer.

3. The ordering conditioner according to claim 1, wherein an outer side of said second bowl is covered with an isolation layer.

4. The ordering conditioner according to claim 1, wherein said first filler is made of far infrared material.

5. The ordering conditioner according to claim 1, wherein said second filler is made of far infrared material.

6. The ordering conditioner according to claim 1, wherein said connection portion is made of non-magnetic material.

7. The ordering conditioner according to claim 1, wherein said first bowl is made of magnetic material.

8. The ordering conditioner according to claim 1, wherein said second bowl is made of magnetic material.
